(19) United States
(12) Patent Application Publication
(10) Pub. No.: US 2023/0371799 A1
(43) Pub. Date: Nov. 23, 2023

(54) ENDOSCOPE

(71) Applicant: Japan Lifeline Co., Ltd., Tokyo (JP)

(72) Inventor: Yuki KODAMA, Tokyo (JP)

(21) Appl. No.: 18/364,012

(22) Filed: Aug. 2, 2023

Related U.S. Application Data
(63) Continuation of application No. PCT/JP2021/004757, filed on Feb. 9, 2021.

(51) Int. Cl. A61B 1/012; A61B 1/01; A61B 1/00; A61B 1/04

(52) U.S. Cl. CPC A61B 1/012; A61B 1/01; A61B 1/00071; A61B 1/042; A61B 1/00066

(57) ABSTRACT

An endoscope of the disclosure includes: a shaft made of resin and including a distal end flexible portion, the shaft including a camera channel, water channels, and a forceps channel formed therein; a handle mounted on a proximal end side of the shaft; and a camera including a camera head and a cable tube, the camera head being equipped with an imaging element, the camera being disposed removably from the shaft and the handle when inserted into the camera channel and the handle. The shaft has an outer diameter (D) of from 2.8 mm to 4.1 mm, the camera channel has a diameter (d1) of from 0.75 mm to 1.2 mm, and (D−d1) is from 1.95 mm to 3.25 mm.

### CROSS REFERENCE TO RELATED APPLICATIONS

This application is a continuation application of International Application No. PCT/JP2021/004757, filed on Feb. 9, 2021, the entire contents of which are hereby incorporated by reference.

### TECHNICAL FIELD

The disclosure relates to an endoscope, and more particularly to an endoscope including a shaft to be inserted into a body, an operation handle, and a camera.

### BACKGROUND

Conventionally, endoscopes including a shaft to be inserted into a body, an operation handle, and a camera have been known as small endoscopes used for diagnostic treatment of biliary ducts and pancreatic ducts, for example, via a duodenoscope (see Patent Document 1 described below).

As illustrated in FIGS. 1 to 4 of JP 4764417 B, in a shaft (catheter 10) constituting such an endoscope (optical catheter system 8), a camera (optical assembly 40) including optical fibers (optical bundle 34) is disposed, and a forceps channel (working channel 60) and a channel (additional channel 62) used for perfusion/imbibition, for example, are formed. Here, the outer diameter of the shaft constituting the endoscope is approximately from 5 Fr to 12 Fr (from 1.67 mm to 4 mm), and the forceps channel (working channel 60) formed therein is assumed to “have a diameter sufficient to accept up to a 4-Fr working device, such as a retrieval basket device or biopsy forceps.”

### SUMMARY

In small-sized endoscopes used for diagnostic treatment of biliary ducts and pancreatic ducts, for example, since the outer diameter of a shaft constituting the endoscope is small, a sufficient diameter of a forceps channel formed in the shaft cannot be secured. In the endoscope described in the above Patent Document 1, the outer diameter of biological forceps that can be inserted into the forceps channel formed in the shaft is 4 Fr (1.33 mm) or less. Such small-diameter biological forceps are not only much more expensive than general-purpose forceps having an outer diameter of about from 1.75 mm to 1.85 mm, but also have a problem in that satisfactory clinical results cannot be obtained because a sufficient amount of tissue cannot be collected at the time of biopsy with such small-diameter forceps.

In addition to the forceps channel, a channel for disposing a camera, a water channel, and a wire lumen through which an operation wire for deflecting a distal end of the shaft is inserted are formed in the small-diameter shaft constituting the endoscope. Therefore, a proportion of resin constituting the shaft is low, the shaft does not have sufficient rigidity, and kinking or the like is likely to occur in a distal end flexible portion of the shaft. When kinking occurs in the distal end flexible portion, optical fibers disposed inside the shaft may be disconnected.

The disclosure has been made in view of such circumstances.

An object of the disclosure is to provide a small-sized endoscope that can be used for diagnostic treatment of biliary ducts and pancreatic ducts, for example, and allows the use of general-purpose biological forceps.

Another object of the disclosure is to provide an endoscope in which kinking is unlikely to occur in a distal end flexible portion of a shaft, and optical fibers constituting a light guide mechanism do not get disconnected.

(1) An endoscope of the disclosure includes: a shaft made of resin and including a distal end flexible portion, the shaft including a camera channel and a forceps channel formed therein; a handle mounted on a proximal end side of the shaft; and a camera including a camera head and a cable tube, the camera head being equipped with an imaging element, the camera being disposed removably from the shaft and the handle when inserted into the camera channel and the handle, wherein respective central axes of the camera channel and the forceps channel are located on the same plane including a central axis of the shaft, the shaft has an outer diameter (D) of from 2.8 mm to 4.1 mm, the camera channel has a diameter (d1) of from 0.75 mm to 1.2 mm, and (D−d1) is from 1.95 mm to 3.25 mm.

With the endoscope having such a configuration, since the outer diameter (D) of the shaft is from 2.8 mm to 4.1 mm, the shaft can be inserted into a biliary duct or a pancreatic duct, for example, to be used for diagnostic treatment in the duct.

In addition, since the difference (D−d1) between the outer diameter (D) of the shaft and the diameter (d1) of the camera channel is from 1.95 mm to 3.25 mm, it is possible to form in the shaft a forceps channel into which general-purpose biological forceps can be inserted.

In addition, since the diameter (d1) of the camera channel is from 0.75 mm to 1.1 mm, which is smaller than that of a conventional endoscope, it is possible to secure a proportion of the resin constituting the shaft to some extent. As a result, it is possible to prevent kinking from occurring in the distal end flexible portion of the shaft.

(2) In the endoscope of the disclosure, the forceps channel preferably has a diameter (d2) of from 1.8 mm to 3.1 mm.

Thus, it is possible to insert general-purpose biological forceps having an outer diameter of about 1.75 mm.

(3) In the endoscope of the disclosure, the forceps channel more preferably has a diameter (d2) of from 1.9 mm to 2.1 mm.

Thus, it is possible to insert general-purpose biological forceps having an outer diameter of about 1.85 mm.

(4) In the endoscope of the above (2) or (3), the camera preferably includes a light guide mechanism including an optical fiber.

With the endoscope having such a configuration, it is not necessary to separately form a channel for disposing the optical fiber, and thus it is possible to sufficiently reduce the diameter of the shaft and the size of the device.

In addition, the camera constituting the endoscope of the disclosure is disposed removably from the shaft (not fixed to the shaft). Thus, when the distal end flexible portion of the shaft is bent, the camera moves inside the camera channel in an axial direction, which makes it possible to lessen a load on the optical fiber located inside the camera.

(5) In a horizontal cross-sectional view of the shaft of the endoscope of the above (4), a proportion of a total area of all channels or lumens including the camera channel and the forceps channel to an area of the shaft is preferably 65% or less (the proportion of resin constituting the shaft being 35% or more).

With the endoscope having such a configuration, since rigidity (bending rigidity) of the shaft can be secured to some extent, it is possible to effectively prevent kinking in the distal end flexible portion.

(6) In the horizontal cross-sectional view of the shaft of the endoscope of the above (5), a total area of all the channels or lumens in a semicircular portion (a remaining portion located in the semicircular portion when the channels and/or lumens partially protrude from the semicircular portion) on a side on which the camera channel is located to an area of the semicircular portion is preferably 58% or less (the proportion of resin constituting the shaft being 42% or more).

With the endoscope having such a configuration, since the rigidity of the shaft in the semicircular portion where the camera (optical fiber) is located can be increased to prevent kinking, it is possible to reliably prevent disconnection of the optical fiber when the distal end flexible portion of the shaft is bent.

(7) In the endoscope of the above (4) to (6), it is preferable that two water channels having a diameter (d3) of from 0.4 mm to 1.0 mm and four wire lumens having a diameter (d4) of from 0.2 mm to 0.5 mm are formed in the shaft, an operation wire including a pullable tail end extends in each of the wire lumens, and pulling the tail end deflects a distal end of the shaft.

(8) The endoscope of the above (7) preferably includes: a handle disposed on the proximal end side of the shaft and including a rotational operation portion; a distal end tip made of resin, disposed on a distal end side of the shaft, and having an outer diameter substantially equal to that of the shaft, the distal end tip including a camera channel, a forceps channel, and water channels formed therein that are in communication with the camera channel, the forceps channel, and the water channels of the shaft, respectively, and that are open at a distal end surface of the distal end tip; an intermediate member made of metal or made of ceramic, disposed between the shaft and the distal end tip, and having a plate shape with an outer diameter substantially equal to that of the shaft, the intermediate member including a main through-hole and four sub-through-holes, the main through-hole being formed surrounding and securing all of communication paths through which the camera channel, the forceps channel, and the water channels of the shaft communicates with the camera channel, the forceps channel, and the water channels of the distal end tip, respectively, the four sub-through-holes being formed corresponding to positions in which the wire lumens are formed; and the four operation wires each passing through a respective one of the sub-through-holes and extending in a respective one of the wire lumens of the shaft, and each including a distal end large-diameter portion that is embedded in the distal end tip and that has a diameter larger than a diameter of the respective one of the sub-through-holes of the intermediate member.

With the endoscope having such a configuration, the distal ends of the operation wires can be securely fixed to the distal end of the shaft by the intermediate member disposed between the shaft made of resin and the distal end tip made of resin. In other words, when the tail end of the operation wire is pulled, the distal end large-diameter portion of the distal end gets caught on the sub-through-hole of the intermediate member. Therefore, the distal end of the operation wire can be restricted from moving in a proximal end direction. As a result, the distal end flexible portion of the shaft can be reliably warped.

In addition, when the tail ends of the operation wires are pulled, pressing force of the distal end large-diameter portions in the proximal end direction with respect to the distal end surface of the shaft is distributed (concentration of the pressing force is lessened) by the intermediate member. Therefore, even when the proportion of resin constituting the shaft is not high, it is possible to more effectively prevent kinking from occurring in the distal end flexible portion.

(9) The endoscope of the disclosure is preferably used as a baby endoscope inserted into a forceps lumen of a side-view scope such as a duodenoscope.

(10) The endoscope of the disclosure is preferably used for diagnostic treatment of a disease in a biliary duct or a pancreatic duct.

According to the endoscope of the disclosure, although the endoscope is a small-sized endoscope including a shaft having an outer diameter of from 2.8 mm to 4.1 mm, it is possible to use general-purpose biological forceps that cannot be used in conventional small-diameter endoscopes.

In addition, kinking is less likely to occur in the distal end flexible portion of the shaft, and it is possible to prevent the optical fiber constituting the light guide mechanism from being disconnected.

### BRIEF DESCRIPTION OF DRAWINGS

FIG. 1 is an explanatory view illustrating an outer appearance of an embodiment of an endoscope of the disclosure.

FIG. 2 is a partially enlarged view (detail view of portion II) illustrating a distal end portion of the endoscope illustrated in FIG. 1.

FIG. 3A is a view taken in the direction of the arrows IIIA-IIIA in FIG. 2.

FIG. 3B is a cross-sectional view taken along the IIIB-IIIB in FIG. 2 (a cross-sectional view of a shaft).

FIG. 3C is a cross-sectional view taken along the IIIC-IIIC in FIG. 2 (a cross-sectional view of a distal end tip).

FIG. 3D is a cross-sectional view taken along the IIID-IIID in FIG. 2 (a cross-sectional view of an intermediate member).

FIG. 4A is a cross-sectional view of a shaft constituting the endoscope illustrated in FIG. 1.

FIG. 4B is a cross-sectional view of the shaft constituting the endoscope illustrated in FIG. 1, indicating in solid lines a semicircular portion on a side on which a camera channel is located.

FIG. 5 is a cross-sectional view schematically illustrating an inside of a camera head.

FIG. 6A is a perspective view illustrating a distal end portion of the endoscope illustrated in FIG. 1 (a camera is not illustrated).

FIG. 6B is a perspective view illustrating the distal end portion of the endoscope illustrated in FIG. 1 (the camera is not illustrated).

FIG. 7A is a cross-sectional view illustrating a state where a slide member of a camera connector constituting the endoscope illustrated in FIG. 1 is at a proximal end position.

FIG. 7B is a cross-sectional view illustrating a state where the slide member of the camera connector constituting the endoscope illustrated in FIG. 1 is at a distal end position.

FIG. 8A is a perspective view illustrating a state where a distal end of the camera constituting the endoscope illustrated in FIG. 1 is at a first position on a proximal end side of a distal end surface of the distal end tip.

FIG. 8B is a perspective view illustrating a state where the distal end of the camera constituting the endoscope illustrated in FIG. 1 is at a second position on a distal end side of the distal end surface of the distal end tip.

### DESCRIPTION OF EMBODIMENTS

### Embodiments

An embodiment of the disclosure will be described.

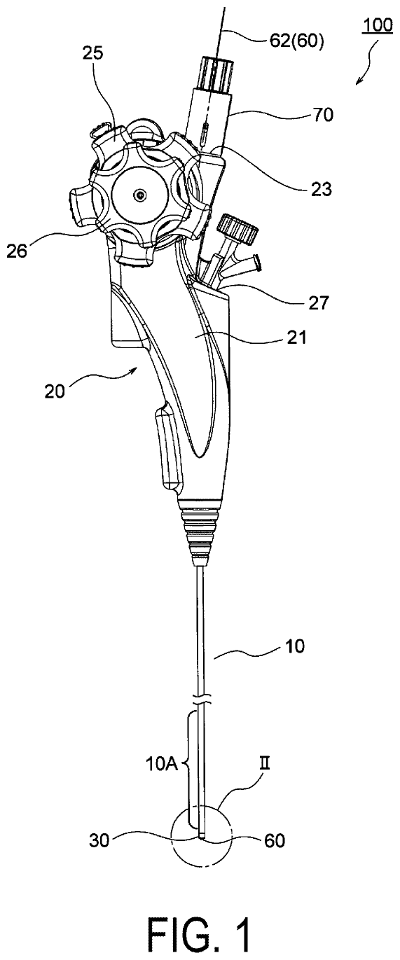

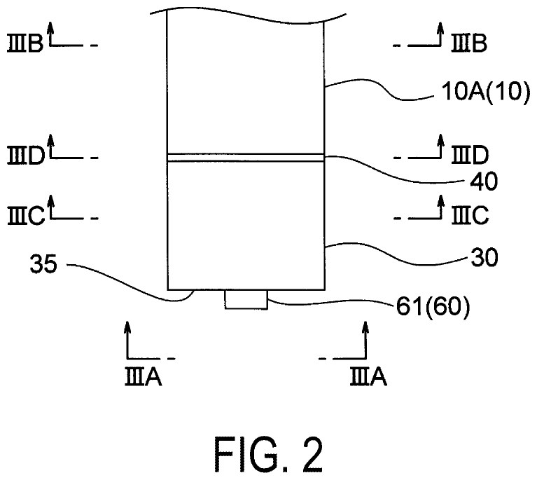

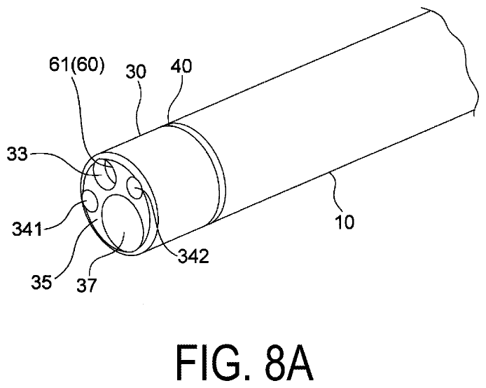

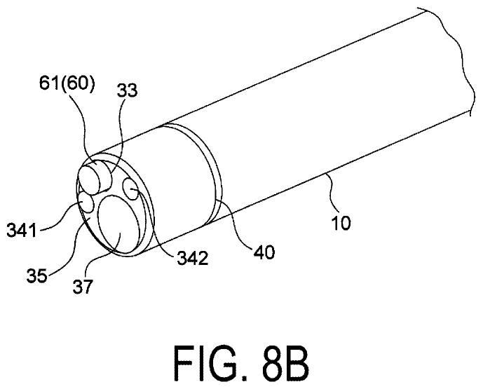

An endoscope 100 of this embodiment illustrated in FIGS. 1 to 8 (FIGS. 8A and 8B) is inserted into a forceps lumen of a side-view scope such as a duodenoscope, and used for diagnostic treatment of diseases in biliary ducts or pancreatic ducts.

This endoscope 100 includes: a shaft 10 made of resin and including a distal end flexible portion 10A, the shaft 10 including a camera channel 13, two water channels 141, 142, and a forceps channel 17 formed therein, as well as four wire lumens 151, 152, 153, 154 formed therein; a handle 20 disposed on a proximal end side of the shaft 10 and including a rotational operation portion (an operation knob 25 and an operation knob 26); a distal end tip 30 made of resin, disposed on a distal end side of the shaft 10, and having an outer diameter equal to that of the shaft 10, the distal end tip 30 including a camera channel 33, water channels 341, 342, and a forceps channel 37 formed therein that are in communication with the camera channel 13, the water channels 141, 142, and the forceps channel 17 of the shaft 10, respectively, and that are open at a distal end surface 35 of the distal end tip; an intermediate member 40 made of metal, disposed between the shaft 10 and the distal end tip 30, and having a disk shape with an outer diameter equal to that of the shaft 10, the intermediate member 40 including a main through-hole 41 and four sub-through-holes 421, 422, 423, 424, the main through-hole 41 being formed surrounding and securing all of communication paths 43, 441, 442, 47 through which the camera channel 13, the water channels 141, 142, and the forceps channel 17 of the shaft 10 communicates with the camera channel 33, the water channels 341, 342, and the forceps channel 37 of the distal end tip 30, respectively, the four sub-through-holes 421, 422, 423, 424 being formed corresponding to positions in which the wire lumens 151, 152, 153, 154 of the shaft 10 are formed; four operation wires 51, 52, 53, 54 each passing through a respective one of the sub-through-holes 421, 422, 423, 424 and extending in a respective one of the wire lumens 151, 152, 153, 154 of the shaft 10, the four operation wires 51, 52, 53, 54 including distal end large-diameter portions 511, 521, 531, 541 that are embedded in the distal end tip 30 and that have diameters larger than diameters of the sub-through-holes 421, 422, 423, 424 of the intermediate member 40, respectively, the four operation wires 51, 52, 53, 54 each including a tail end that is fixed to the rotational operation portion (the operation knob 25 or the operation knob 26) of the handle 20 and that can be pulled; and a camera 60 including a camera head 61 and a cable tube 62, the camera head 61 being equipped with a CMOS image sensor 611 (imaging element), the camera 60 including a plurality of optical fibers 65 built therein, the camera 60 being disposed removably from the shaft 10 and the handle 20 when inserted into the camera channel 13 and the handle 20; in which respective central axes of the camera channel 13 and the forceps channel 17 formed in the shaft 10 are located on the same plane including a central axis of the shaft, the shaft 10 has an outer diameter (D) of from 2.8 mm to 4.1 mm, the camera channel 13 has a diameter (d1) of from 0.75 mm to 1.1 mm, and a difference (D−d1) between the outer diameter of the shaft 10 and the diameter of the camera channel is from 1.95 mm to 3.25 mm.

The endoscope 100 includes the shaft 10 to be inserted into the body, the handle 20 disposed on the proximal end side of the shaft 10, the distal end tip 30 disposed on the distal end side of the shaft 10, the intermediate member 40 disposed between the shaft 10 and the distal end tip 30, the operation wires 51, 52, 53, 54, and the camera 60.

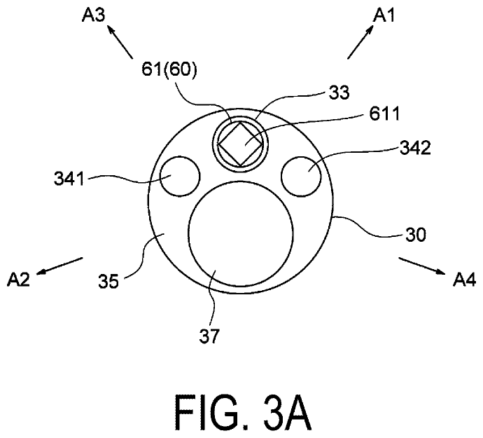

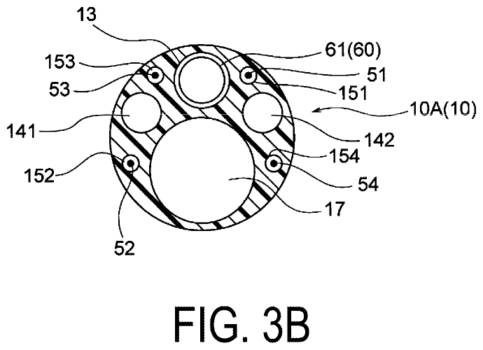

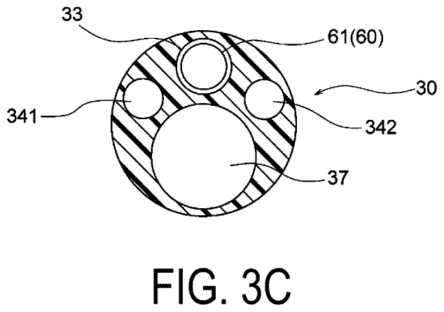

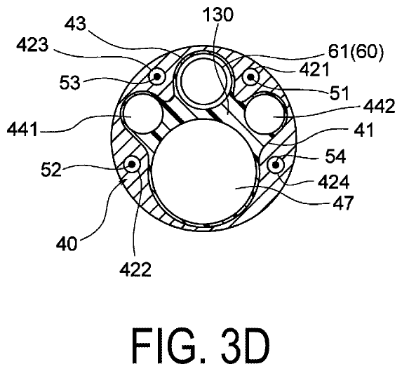

As illustrated in FIGS. 3B, 4A, and 6A, the camera channel 13, the water channels 141, 142, and the forceps channel 17 are formed in the shaft 10 constituting the endoscope 100.

Also, the wire lumens 151, 152, 153, 154 that are insertion paths for the operation wires 51, 52, 53, 54, are formed in the shaft 10.

The length (effective length) of the shaft 10 is preferably 200 mm to 4800 mm, and is 1900 mm in a preferred example.

The shaft 10 includes the distal end flexible portion 10A.

Here, the “distal end flexible portion” refers to a distal end portion of the shaft that can be warped (bent) by pulling the tail end of the operation wire.

The length of the distal end flexible portion 10A is preferably 5 mm to 200 mm, and is 20 mm in a preferred example.

The outer diameter (D) of the shaft 10 typically ranges from 2.8 mm to 4.1 mm, preferably from 3.2 mm to 3.7 mm, and is 3.6 mm in a preferred example.

The shaft 10 having such a small diameter can be inserted into a biliary duct or a pancreatic duct to perform diagnostic treatment in the duct.

The diameter (d1) of the camera channel 13 typically ranges from 0.75 mm to 1.2 mm, preferably from 0.95 mm to 1.1 mm, and is 1.05 mm in a preferred example.

The difference (D−d1) between the outer diameter (D) of the shaft 10 and the diameter (d1) of the camera channel typically ranges from 1.95 mm to 3.25 mm, preferably from 2.05 mm to 3.18 mm, and is 2.3 mm (3.5 mm−1.2 mm) in a preferred example.

Since the difference (D−d1) between the outer diameter (D) of the shaft and the diameter (d1) of the camera channel is from 1.95 mm to 3.25 mm, the diameter (d2) of the forceps channel 17 can have such a size (1.8 mm or more) that general-purpose biological forceps having an outer diameter of 1.75 mm or more can be inserted therethrough.

If (D−d1) were less than 1.95 mm, it would be impossible or very difficult to form a forceps channel through which general-purpose biological forceps can be inserted.

If (D−d1) exceeded 3.25 mm, it would be impossible or very difficult to reduce the outer diameter of the shaft to 4.1 mm or less.

In addition, since the diameter (d1) of the camera channel is from 0.75 mm to 1.1 mm, which is smaller than that of conventional endoscopes, it is possible to secure a proportion of the resin constituting the shaft 10 to some extent. As a result, it is possible to prevent kinking from occurring in the distal end flexible portion 10A of the shaft 10.

If (d1) were less than 0.75 mm, it would be impossible or very difficult to insert the camera.

If (d1) exceeded 1.1 mm, it would be impossible or very difficult to reduce (D−d1) to 3.25 mm or less.

The diameter (d2) of the forceps channel 17 is preferably from 1.8 mm to 3.1 mm, is more preferably from 1.9 mm to 2.1 mm, and is 2.0 mm in a preferred example.

With the diameter (d2) of the forceps channel 17 being 1.8 mm or more, it becomes possible to insert general-purpose biological forceps having an outer diameter of about 1.75 mm that cannot be inserted through conventional endoscopes. With the diameter (d2) of the forceps channel 17 being 1.9 mm or more, it also becomes possible to insert general-purpose biological forceps having an outer diameter of about 1.85 mm.

By using such general-purpose biological forceps, a sufficient amount of tissue can be collected at the time of biopsy, and favorable clinical results can be obtained.

Further, the general-purpose biological forceps are much less expensive than forceps having an outer diameter of 4 Fr (1.33 mm) or less that can be inserted through conventional endoscopes.

The diameter (d3) of the water channels 141, 142 is preferably from 0.4 mm to 1.0 mm, and is 0.75 mm in a preferred example.

The diameters (d4) of the wire lumens 151, 152, 153, 154 are preferably from 0.2 mm to 0.5 mm, and is 0.33 mm in a preferred example.

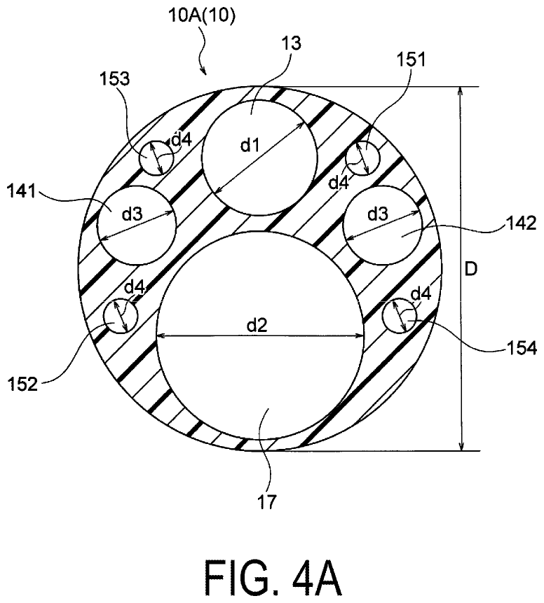

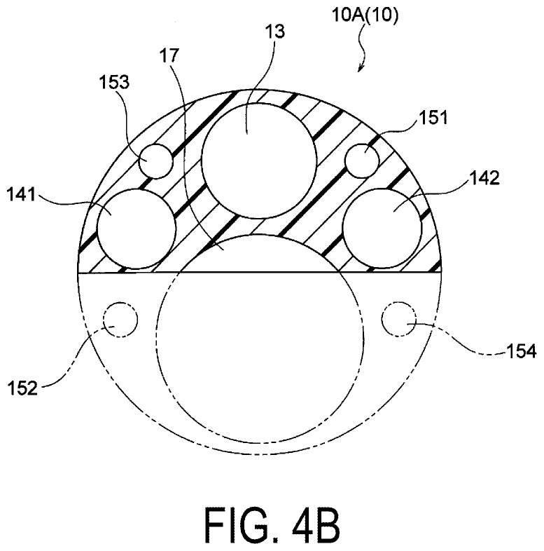

In a horizontal cross-sectional view of the shaft 10 as illustrated in FIG. 4A, a proportion of a total area [π(d1)²/4+π(d2)²/4+π(d3)²/2+π(d4)²] of the camera channel 13, the forceps channel 17, the water channels 141, 142, and the wire lumens 151, 152, 153, 154 to an area of the shaft [π(D)²/4] is preferably 65% or less (the proportion of resin constituting the shaft being 35% or more), more preferably from 50% to 60%, and 55% [(1.1²π/4+2.0²π/4+0.75²π/2+0.33²π/(3.5²π/4)] in a preferred example.

With this proportion being 65% or less (the proportion of resin being 35% or more), the bending rigidity or torsional rigidity of the shaft 10 can be secured to some extent, and it is possible to effectively prevent kinking in the distal end flexible portion 10A.

In a horizontal cross-sectional view of the shaft 10 (a semicircular portion on the side on which the camera channel 13 is located) as illustrated in FIG. 4B, a proportion of the total area of all the channels or lumens in the semicircular portion, that is, the camera channel 13, the water channels 141, 142, the wire lumens 151, 153, and the forceps lumen 17 (excluding regions protruding from the semicircular portion) to the area of the semicircular portion [π(D)²/8] is preferably 58% or less (the proportion of resin constituting the shaft being 42% or more), more preferably from 49% to 53%, and 51% in a preferred example.

With this proportion being 58% or less (the proportion of resin being 42% or more), the rigidity of the shaft 10 in the semicircular portion where the camera 60 (optical fibers 65) is located can be increased to prevent kinking. Therefore, when the distal end flexible portion 10A of the shaft 10 is bent, it is possible to reliably prevent disconnection of the optical fibers 65.

The shaft 10 is made of resin.

Examples of the resin material forming the shaft 10 include nylon resin, polyether block amide (PEBAX) resin, polyurethane resin, and polyolefin resin. Of these resins, PEBAX resin and polyurethane resin are preferable.

The hardness (Shore D hardness) of the constituent resin of the shaft 10 is preferably 90 D or less. In a preferred example, the hardness of the resin forming the distal end flexible portion 10A is 25 D, and the hardness of the resin forming a portion other than the distal end flexible portion 10A is 30 D.

The handle 20 is disposed on the proximal end side of the shaft 10.

The handle 20 constituting the endoscope 100 includes a grip 21, and the two operation knobs 25, 26 as a rotational operation portion.

The handle 20 is provided with a camera channel port 23 in communication with the camera channel 13, and a forceps channel port 27 in communication with the forceps channel 17.

The distal end tip 30 is disposed on the distal end side of the shaft 10.

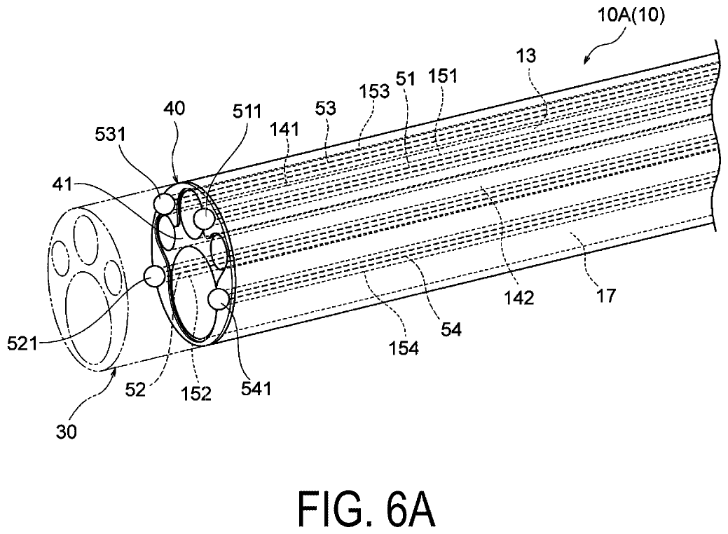

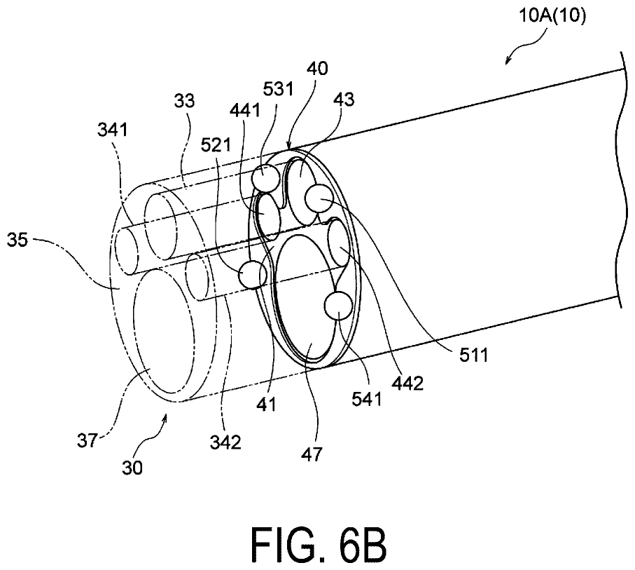

As illustrated in FIGS. 3A, 3C, and 6B, the camera channel 33, the water channels 341, 342, and the forceps channel 37 are formed in the distal end tip 30 constituting the endoscope 100.

The camera channel 33 is in communication with the camera channel 13 of the shaft 10 via the communication path 43. The diameter of the camera channel 33 is equal to the diameter of the camera channel 13 in communication with the camera channel 33.

The water channels 341, 342 are each in communication with the water channels 141, 142 of the shaft 10 via the communication paths 441, 442. The diameter of the water channels 341, 342 is equal to the diameter of the water channels 141, 142 in communication with the water channels 341, 342.

The forceps channel 37 is in communication with the forceps channel 17 of shaft 10 via the communication path 47. The diameter of the forceps channel 37 is equal to the diameter of the forceps channel 17 in communication with the forceps channel 37.

The length of the distal end tip 30 is preferably 1 mm to 30 mm, and is 3 mm in a preferred example.

The outer diameter of the distal end tip 30 is equal to the outer diameter of the shaft 10.

The distal end tip 30 is made of resin.

Examples of the resin material forming the distal end tip 30 include resins similar to those illustrated as the resins forming the shaft 10, and of these resins, PEBAX resin and polyurethane resin are preferable.

The distal end tip 30 is made of a resin material having low hardness so as not to damage body tissue. The hardness (Shore D hardness) of the constituent resin of the distal end tip 30 is preferably 72 D or less, and is 25 D in a preferred example.

The intermediate member 40 having a disk shape is disposed between the shaft 10 and the distal end tip 30.

The intermediate member 40 constituting the endoscope 100 is a member for fixing a distal end of each of the operation wires 51, 52, 53, 54 to a distal end of the shaft 10 (for preventing removal of the tail ends during the pulling operation).

As illustrated in FIGS. 3D and 6B, one main through-hole 41 that surrounds all of the communication path 43, the communication paths 441, 442, and the communication path 47 is formed in the intermediate member 40.

Here, the communication path 43 is a path (camera channel) defined and formed by a constituent resin 130 of the shaft 10 and/or the distal end tip 30 in order to allow the camera channel 13 of the shaft 10 to communicate with the camera channel 33 of the distal end tip 30. The diameter of the communication path 43 is equal to the diameters of the camera channel 13 and the camera channel 33.

The communication paths 441, 442 are paths (water channels) defined and formed by the constituent resin 130 of the shaft 10 and/or the distal end tip 30 in order to allow the water channels 141, 142 of the shaft 10 to respectively communicate with the water channels 341, 342 of the distal end tip 30. The diameter of the communication paths 441, 442 is identical to the diameters of the water channels 141, 142 and the water channels 341, 342.

The communication path 47 is a path (forceps channel) defined and formed by the constituent resin 130 of the shaft 10 and/or the distal end tip 30 to allow the forceps channel 17 of the shaft 10 to communicate with the forceps channel 37 of the distal end tip 30. The diameter of the communication path 47 is equal to the diameters of the forceps channel 17 and the forceps channel 37.

As illustrated in FIG. 3D, in the intermediate member 40, four sub-through-holes 421, 422, 423, 424 are formed corresponding to the positions in which the wire lumens 151, 152, 153, 154 of the shaft 10 are formed. The sub-through-holes 421, 422, 423, 424 are insertion paths for the operation wires 51, 52, 53, 54.

The sub-through-holes 421, 422, 423, 424 are circular holes, and the diameter of each of the sub-through-holes is adjusted to be larger than the diameter of the operation wires 51, 52, 53, 54 to be inserted therethrough and smaller than the diameter of the distal end large-diameter portions 511, 521, 531, 541 to be restricted from being inserted therethrough.

The diameter of the sub-through-hole is preferably 0.13 mm to 2.5 mm, and is 0.35 mm in a preferred example.

The thickness of the intermediate member 40 is preferably 0.05 mm to 3 mm, and is 0.15 mm in a preferred example.

If the thickness of the intermediate member 40 is too small, the intermediate member 40 may be damaged upon mechanical shock associated with the pulling operation of the operation wires 51, 52, 53, 54.

On the other hand, if the thickness is too large, the intermediate member 40 itself is less likely to warp, and thus the distal end flexible portion 10A may be difficult to warp.

The outer diameter of the intermediate member 40 is equal to the outer diameters of the shaft 10 and the distal end tip 30, and thus the outer peripheral surface of the shaft 10, the outer peripheral surface of the intermediate member 40, and the outer peripheral surface of the distal end tip 30 are flush with one another. Accordingly, the intermediate member 40 does not protrude from between the shaft 10 and the distal end tip 30 to expose the edge of the intermediate member 40, and thus body tissue and the like are not damaged by such an edge.

The intermediate member 40 is made of metal or ceramic, and is preferably made of metal.

Examples of the metal material forming the intermediate member 40 include stainless copper, platinum, gold, copper, nickel, titanium, and tantalum. Of these metals, stainless copper is preferable.

In the endoscope 100 of the present embodiment, the shaft 10 and the distal end tip 30 are directly bonded to each other (the constituent resins of the shaft 10 and the distal end tip 30 are welded to each other) in a region on an inner side of the main through-hole 41 of the intermediate member 40 and on the outer side of each of the communication paths 43, 441, 442, 47 (the region surrounded by the main through-hole 41 excluding the communication paths). As a result, the intermediate member 40 is also secured with the resin present on the inner side of the main through-hole 41, and the intermediate member 40 is firmly secured to the shaft 10 and the distal end tip 30.

Also, the shaft 10 and distal end tip 30 are directly bonded to each other, albeit partially, and thus the fixing strength of the distal end tip 30 with respect to the shaft 10 is also sufficiently increased.

Here, the value of (S)/(S₀) is preferably 0.1 or greater, and is more preferably from 0.3 to 0.6, where (S) is an area of the region in which the constituent resin of the shaft 10 and the distal end tip 30 are directly bonded to each other, and (S₀) is a cross-sectional area of the shaft 10.

As illustrated in FIGS. 3B and 6A, the operation wires 51, 52, 53, 54 extend in the wire lumens 151, 152, 153, 154 of the shaft 10, respectively.

As illustrated in FIGS. 6A and 6B, the distal ends of the operation wires 51, 52, 53, 54 are the distal end large-diameter portions 511, 521, 531, 541, respectively.

The distal end large-diameter portions 511, 521, 531, 541 have spherical or partially spherical shapes with a diameter larger than the diameter of the sub-through-holes 421, 422, 423, 424 of the intermediate member 40, and cannot pass through the sub-through-holes 421, 422, 423, 424.

The diameter of the distal end large-diameter portions 511, 521, 531, 541 is preferably 0.2 mm to 3.5 mm, and is 0.4 mm in a preferred example.

The diameter of the operation wires 51, 52, 53, 54 (a portion other than the distal end large-diameter portion) is preferably 0.1 mm to 2.0 mm, and is 0.25 mm in a preferred example.

The operation wires 51, 52 are held in a state where each of the distal end large-diameter portions 511, 521 is embedded in the distal end tip 30. Wire portions located on the proximal end side of the distal end large-diameter portions 511, 521 pass through the sub-through-holes 421, 422 of the intermediate member 40 and extend in the wire lumens 151, 152 of the shaft 10, respectively. Each of the proximal ends of the operation wires 51, 52 is fixed to the operation knob 25 of the handle 20.

Rotating the operation knob 25 in one direction and pulling the proximal end of the operation wire 51 causes the operation wire 51 to move the wire lumen 151 in a proximal end direction. At this time, the distal end large-diameter portion 511 gets caught on the sub-through-hole 421 of the intermediate member 40 to be restricted from moving in the proximal end direction. Therefore, the distal end flexible portion 10A of the shaft 10 warps in the direction indicated by arrow A1 in FIG. 3A, and the distal end of the endoscope 100 (the distal end tip 30) deflects in the same direction.

Rotating the operation knob 25 in the other direction and pulling the proximal end of the operation wire 52 causes the operation wire 52 to move the wire lumen 152 in the proximal end direction. At this time, the distal end large-diameter portion 521 gets caught on the sub-through-hole 422 of the intermediate member 40 to be restricted from moving in the proximal end direction. Therefore, the distal end flexible portion 10A of the shaft 10 warps in the direction indicated by arrow A2 in FIG. 3A, and the distal end of the endoscope 100 (the distal end tip 30) deflects in the same direction.

The operation wires 53, 54 are held in a state where each of the distal end large-diameter portions 531, 541 is embedded in the distal end tip 30. Wire portions located on the proximal end side of the distal end large-diameter portions 531, 541 pass through the sub-through-holes 423, 424 of the intermediate member 40 and extend in the wire lumens 153, 154 of the shaft 10, respectively. Each of the proximal ends of the operation wires 53, 54 is fixed to the operation knob 26 of the handle 20.

Rotating the operation knob 26 in one direction and pulling the proximal end of the operation wire 53 causes the operation wire 53 to move the wire lumen 153 in the proximal end direction. At this time, the distal end large-diameter portion 531 gets caught on the sub-through-hole 423 of the intermediate member 40 to be restricted from moving in the proximal end direction. Therefore, the distal end flexible portion 10A of the shaft 10 warps in the direction indicated by arrow A3 in FIG. 3A, and the distal end of the endoscope 100 (the distal end tip 30) deflects in the same direction.

Rotating the operation knob 26 in the other direction and pulling the proximal end of the operation wire 54 causes the operation wire 54 to move the wire lumen 154 in the proximal end direction. At this time, the distal end large-diameter portion 541 gets caught on the sub-through-hole 424 of the intermediate member 40 to be restricted from moving in the proximal end direction. Therefore, the distal end flexible portion 10A of the shaft 10 warps in the direction indicated by arrow A4 in FIG. 3A, and the distal end of the endoscope 100 (the distal end tip 30) deflects in the same direction.

When the tail ends of the operation wires 51, 52, 53, 54 are pulled as described above, the distal end large-diameter portions 511, 521, 531, 541 get caught on the sub-through-holes 421, 422, 423, 424 formed in the intermediate member 40, respectively, and the distal ends of the operation wires 51, 52, 53, 54 are fixed to the distal end of the shaft 10 (are prevented from being removed). Thus, the distal end flexible portion 10A of the shaft 10 can be warped in intended directions (the directions indicated by arrows A1 to A4).

If the intermediate member were not disposed between the shaft and the distal end tip, pulling the proximal end of the operation wire might cause the distal end large-diameter portion to move in the proximal end direction while pushing and expanding the wire lumen, with the distal end of the operation wire (the distal end large-diameter portion) being not sufficiently fixed with respect to the distal end of the shaft (being not prevented from being removed). In such a case, the distal end flexible portion could not be warped.

The constituent material of the operation wires 51, 52, 53, 54 is not particularly limited, and the same material as the constituent material of operation wires used in conventionally known medical devices that can perform distal end deflection operation can be used.

The camera 60 constituting the endoscope 100 includes the camera head 61 equipped with the CMOS image sensor 611, and the cable tube 62 containing a transmission cable of the CMOS image sensor 611.

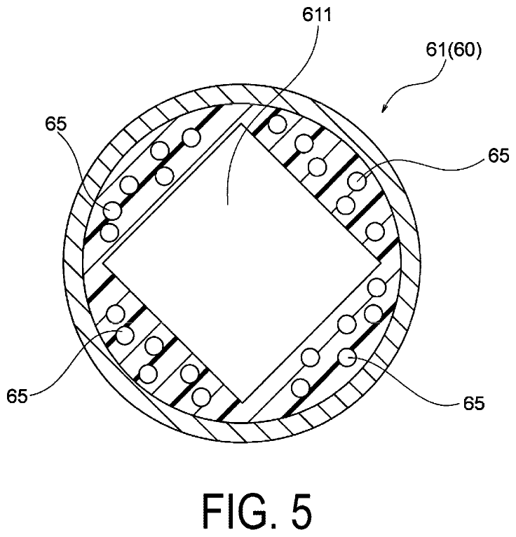

As illustrated in FIG. 5, the camera 60 includes a plurality of (24 in the illustrated example) optical fibers 65 built therein and surrounding the CMOS image sensor 611.

This eliminates the need to separately form a channel for disposing the optical fibers, and it is possible to sufficiently reduce the diameter of the shaft 10 and the size of the device.

In addition, the camera 60 constituting the endoscope 100 is disposed removably from the shaft 10 (not fixed to the shaft 10). Thus, when the distal end flexible portion 10A of the shaft 10 is bent, the camera 60 moves inside the camera channel 13 in an axial direction, which makes it possible to lessen a load on the optical fibers 65 located inside the camera 60.

The outer diameter of the camera head 61 is preferably from 0.7 mm to 1.0 mm, and is 1.0 mm in a preferred example. The outer diameter of the cable tube 62 is substantially equal to the outer diameter of the camera head 61.

The camera 60 is disposed in the camera channels (the camera channel 13 and the camera channel 33) of the shaft 10 and the distal end tip 30. A proximal end portion of the cable tube 62 protrudes out of the camera channel port 23 of the handle 20, and a proximal end of the cable tube is connected to a control device.

A camera connector 70 is attached to the cable tube 62 of the camera 60.

The camera connector 70 is mounted to the camera channel port 23 of the handle 20 when the camera 60 is properly disposed in the camera channel 13 and the camera channel 33.

In other words, mounting the camera connector 70 to the camera channel port 23 causes the camera 60 to be properly disposed in the camera channel 13 and the camera channel 33.

The attaching position of the camera connector 70 is 300 to 5000 mm from a distal end of the camera 60, and is 2100 mm from the distal end of the camera 60 in a preferred example.

In the endoscope 100 of the present embodiment, the camera 60 is separable from the handle 20 and the shaft 10.

In other words, with the camera connector 70 removed from the camera channel port 23, the camera 60 disposed in the camera channel 13 and the camera channel 33 can be removed from the camera channel port 23 of the handle 20 along with the camera connector 70.

Also, once separated, the camera 60 can be reincorporated as a component of the endoscope 100 by inserting, with the camera head 61 in front, the camera 60 from the camera channel port 23 of the handle 20 into the handle 20 and the camera channel 13 of the shaft 10, and mounting the camera connector 70 to the camera channel port 23.

The camera connector 70 includes a camera position adjustment mechanism that allows, when camera connector 70 is mounted to the camera channel port 23, the camera 60 to reciprocate with respect to the camera channel 13 and the camera channel 33 such that a distal end of the camera 60 disposed in the camera channel 13 and the camera channel 33 is displaced between a first position (a distal end position of the camera 60 as illustrated in FIG. 8A) located on the proximal end side of the distal end surface 35 of the distal end tip 30 at which the camera channel 33 opens and a second position (a distal end position of the camera 60 as illustrated in FIG. 8B) located on the distal end side of the distal end surface 35.

Here, the distance from the first position to the second position (the movement distance of the distal end of the camera 60 by the position adjustment mechanism) is preferably 2 mm to 100 mm, and is 30 mm in a preferred example.

In addition, the distance from the distal end surface 35 of the distal end tip 30 to the first position is preferably 1.5 mm to 20 mm, and the distance from the distal end surface 35 to the second position is preferably 0.5 mm to 80 mm.

In the endoscope 100 of the present embodiment, the camera position adjustment mechanism included in the camera connector 70 is a mechanism that reciprocates the camera 60 by using a feed screw.

Specifically, the mechanism includes: a connector case 71 mounted to the camera channel port 23 and including an inner peripheral surface on which a guide groove (not illustrated) extending in the axial direction is formed and a peripheral wall on which a guide hole 713 extending in the axial direction is formed; a slide member 72 slidable with respect to the connector case 71, the slide member 72 including a shaft portion 721 and a guide portion 723, the shaft portion 721 extending within the connector case 71 and including a portion protruding toward the proximal end side of the connector case 71, the shaft portion 721 including a proximal end portion on which an external threaded portion 722 is formed, the shaft portion 721 being adhesively fixed to the cable tube 62 of the camera 60 with the cable tube 62 inserted therein, the guide portion 723 being integrally formed with the shaft portion 721 and surrounding a distal end portion of the shaft portion 721, the guide portion 723 including an outer peripheral surface on which a projecting portion (not illustrated) to be guided by the guide groove of the connector case 71 is formed and an outer peripheral side on which a protruding portion 725 to be guided by the guide hole 713 is formed; and a rotation knob 73 located on the proximal end side of the connector case 71 and being restricted from moving in the axial direction, the rotation knob 73 including an internal threaded portion 731 that screws with the external threaded portion 722 of the shaft portion 721 of the slide member 72; in which rotating the rotation knob 73 in one direction to slide the slide member 72 from a proximal end position to a distal end position moves the distal end of the camera 60 from the first position (the distal end position of the camera 60 as illustrated in FIG. 8A) to the second position (the distal end position of the camera 60 as illustrated in FIG. 8B), and rotating the rotation knob 73 in the other direction to slide the slide member 72 from the distal end position to the proximal end position moves the distal end of the camera 60 from the second position to the first position.

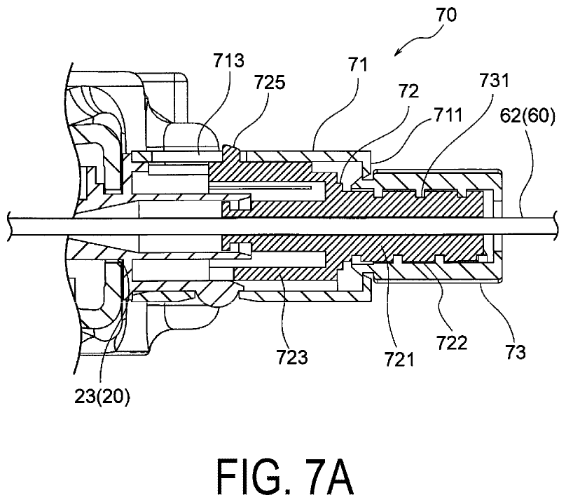

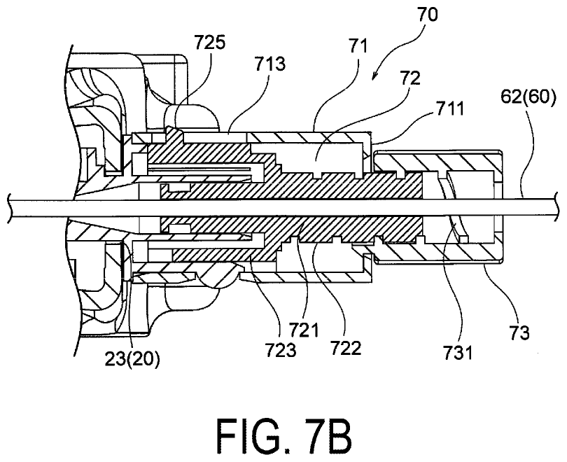

Here, the “proximal end position” is a position in which the slide member 72 cannot move from that position further toward the proximal end side as illustrated in FIG. 7A, and the “distal end position” is a position in which the slide member 72 cannot move from that position further toward the distal end side as illustrated in FIG. 7B.

The camera position adjustment mechanism includes the connector case 71, the slide member 72, and the rotation knob 73.

The connector case 71 is a constituent member of the camera connector 70 mounted to the camera channel port 23 via a port-side connector described below, and includes a tubular body having an arch-shaped portion.

The guide groove extending in the axial direction is formed on the inner peripheral surface of the connector case 71, and the guide hole 713 extending in the axial direction is formed in the peripheral wall of the arch-shaped portion.

The slide member 72 includes the shaft portion 721, and the guide portion 723 that is integrally formed with the shaft portion 721 and that surrounds the distal end portion of the shaft portion 721.

The shaft portion 721 of the slide member 72 extends within the connector case 71, and a portion of the shaft portion 721 protrudes out of an opening formed in a proximal end surface 711 of the connector case 71 toward the proximal end side.

The external threaded portion 722 is formed on the proximal end portion of the shaft portion 721.

As illustrated in FIGS. 7A and 7B, the cable tube 62 of the camera 60 is adhesively fixed inside the shaft portion 721 while being inserted therethrough.

The guide portion 723 of the slide member 72 includes an arch-shaped portion matching the shape of the connector case 71, is integrally formed with the shaft portion 721, and surrounds the distal end portion of the shaft portion 721.

The projecting portion to be guided by the guide groove of the connector case 71 is formed on the outer peripheral surface of the guide portion 723.

Additionally, the protruding portion 725 to be guided by the guide hole 713 of the connector case 71 is formed on the outer peripheral side of the arch-shaped portion of the guide portion 723.

The rotation knob 73 is disposed on the proximal end side of the connector case 71.

The internal threaded portion 731 that screws with the external threaded portion 722 of the shaft portion 721 of the slide member 72 is formed on the inner peripheral side of the rotation knob 73.

The rotation knob 73 is restricted from moving in the axial direction with respect to the connector case 71. Rotating the rotation knob 73 slides the slide member 72 with respect to the connector case 71.

Additionally, rotating the rotation knob 73 to slide the slide member 72 also moves the cable tube 62 adhesively fixed inside the shaft portion 721 in the axial direction with respect to the connector case 71.

With the camera position adjustment mechanism having such a configuration as described above, rotating the rotation knob 73 in one direction to slide the slide member 72 from the proximal end position (the position illustrated in FIG. 7A) to the distal end position (the position illustrated in FIG. 7B) can move the distal end of the camera 60 from the first position (the distal end position of the camera 60 as illustrated in FIG. 8A) to the second position (the distal end position of the camera 60 as illustrated in FIG. 8B). Rotating the rotation knob 73 in the other direction to slide the slide member 72 from the distal end position to the proximal end position can move the distal end of the camera 60 from the second position to the first position.

According to the endoscope 100 of the present embodiment, since the outer diameter (D) of the shaft 10 is from 2.8 mm to 4.1 mm, the shaft 10 can be inserted into a biliary duct or a pancreatic duct, for example, to be used for diagnostic treatment in the duct.

In addition, since the difference (D−d1) between the outer diameter (D) of the shaft 10 and the diameter (d1) of the camera channel 13 is from 1.95 mm to 3.25 mm, the diameter (d2) of the forceps channel 17 can be 1.8 mm or more, preferably 1.9 mm or more, so that general-purpose biological forceps can be inserted therethrough. In addition, since the diameter (d1) of the camera channel 13 is from 0.75 mm to 1.1 mm, which is smaller than that of conventional endoscopes, it is possible to secure the proportion of resin constituting the shaft 10 to some extent. As a result, it is possible to prevent kinking from occurring in the distal end flexible portion of the shaft.

In addition, in a horizontal cross-sectional view of the shaft 10, with a proportion of the total area of the camera channel 13, the forceps channel 17, the water channels 141, 142, and the wire lumens 151, 152, 153, 154 to the area of the shaft 10 being 65% or less, the bending rigidity of the shaft 10 can be secured to some extent, and it is possible to effectively prevent kinking in the distal end flexible portion 10A.

In addition, in the horizontal cross-sectional view of the shaft 10, with a proportion of the total area of the camera channel 13, the water channels 141, 142, the wire lumens 151, 153, and the forceps lumen 17 (excluding the region protruding from the semicircular portion) in the semicircular portion on the side on which the camera channel 13 is located to the area of the semicircular portion being 58% or less, the rigidity of the shaft 10 in the semicircular portion where the camera 60 (optical fibers 65) is located can be increased to prevent kinking. Therefore, when the distal end flexible portion 10A of the shaft 10 is bent, it is possible to reliably prevent disconnection of the optical fibers 65.

In addition, the distal ends of the operation wires 51, 52, 53, 54 can be securely fixed to the distal end of the shaft 10 by the intermediate member 40 made of metal and disposed between the shaft 10 and the distal end tip 30. In other words, when the tail ends of the operation wires 51, 52, 53, 54 are pulled, the distal end large-diameter portions 511, 521, 531, 541 get caught on the sub-through-holes 421, 422, 423, 424 of the intermediate member 40, respectively. Therefore, the distal ends of the operation wires 51, 52, 53, 54 can be restricted from moving in the proximal end direction. Thus, the distal end flexible portion 10A of the shaft 10 can be reliably warped in the intended direction.

In addition, when the tail ends of the operation wires 51, 52, 53, 54 are pulled, pressing force of the distal end large-diameter portions 511, 521, 531, 541 in the proximal end direction with respect to the distal end surface of the shaft 10 is distributed (concentration of the pressing force is lessened) by the intermediate member 40. Therefore, it is possible to more effectively prevent kinking from occurring in the distal end flexible portion 10A.

Further, the outer diameter of the intermediate member 40 is equal to the outer diameters of the shaft 10 and the distal end tip 30, and thus the outer peripheral surface of the shaft 10, the outer peripheral surface of the intermediate member 40, and the outer peripheral surface of the distal end tip 30 are flush with one another. Accordingly, the intermediate member 40 does not protrude from between the shaft 10 and the distal end tip 30 to expose the edge of the intermediate member 40, and thus body tissue and the like are not damaged by the edge of the intermediate member 40.

Furthermore, since the intermediate member 40 has a disk shape and is located at the distal end of the shaft 10, the entire distal end flexible portion 10A of the shaft 10 can be warped to perform a smooth deflection operation, unlike known endoscopes in which distal ends of operation wires are fixed by a cylindrical metal member.

In addition, the shaft 10 and the distal end tip 30 are directly bonded to each other (the constituent resins of the shaft and the distal end tip are welded to each other) on the inner side of the main through-hole 41 of the intermediate member 40 and on the outer side of each of the communication paths 43, 441, 442, 47 (in a region surrounded by the main through-hole 41 excluding the communication paths). Therefore, the intermediate member 40 is also secured with the resin present on the inner side of the main through-hole 41, and no positional shift is caused that causes the intermediate member 40 to rotate about the axis of the shaft 10.

Moreover, the shaft 10 and the distal end tip 30 are directly bonded to each other on the inner side of the main through-hole 41 of the intermediate member 40 and on the outer side of the communication paths 43, 441, 442, 47. Thus, although the intermediate member 40 made of metal is interposed between the shaft 10 and the distal end tip 30, the distal end tip 30 can be firmly secured to the shaft 10, and the distal end tip 30 does not fall off from the distal end of the shaft 10.

Further, each of the distal end large-diameter portions 511, 521, 531, 541 of the operation wires 51, 52, 53, 54 is embedded in the distal end tip 30. Thus, during the pulling operation of the operation wire 51 or 52, the distal end of the operation wire 52 or 51 opposed to the operation wire 51 or 52 does not move (is not drawn out) in the distal end direction. Also, during the pulling operation of the operation wire 53 or 54, the distal end of the operation wire 54 or 53 opposed to the operation wire 53 or 54 does not move (is not drawn out) in the distal end direction.

Furthermore, unlike a case where a cylindrical metal member is mounted, since the disk-shaped intermediate member 40 is mounted, it is not necessary to cut the outer periphery of the shaft 10. Therefore, even when the shaft 10 has a small diameter, a sufficient diameter of the forceps channel 17 can be secured.

Additionally, by separating, from the handle and the shaft, the camera 60 having an expensive solid state imaging element and washing the camera 60 after use, the camera 60 can be incorporated and reused as a component of the endoscope 100.

Further, with the camera position adjustment mechanism, the shaft 10 can be reliably prevented from being inserted into a delivery device or the body in a state where the camera 60 protrudes from the opening of the camera channel 33 at the distal end surface 35 of the distal end tip 30.

Furthermore, the distal end position of the camera 60 with respect to the distal end surface 35 of the distal end tip 30 can be finely adjusted by the camera position adjustment mechanism that reciprocates the camera 60 by using a feed screw.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An endoscope, comprising:
a shaft made of resin and including a distal end flexible portion, the shaft including a camera channel and a forceps channel formed therein;
a handle mounted on a proximal end side of the shaft; and
a camera including a camera head and a cable tube, the camera head being equipped with an imaging element, the camera being disposed removably from the shaft and the handle when inserted into the camera channel and the handle, wherein
respective central axes of the camera channel and the forceps channel are located on the same plane including a central axis of the shaft,
the shaft has an outer diameter (D) of from 2.8 mm to 4.1 mm,
the camera channel has a diameter (d1) of from 0.75 mm to 1.2 mm, and
(D−d1) is from 1.95 mm to 3.25 mm.

2. The endoscope according to claim 1, wherein
the forceps channel has a diameter (d2) of from 1.8 mm to 3.1 mm.

3. The endoscope according to claim 1, wherein
the forceps channel has a diameter (d2) of from 1.9 mm to 2.1 mm.

4. The endoscope according to claim 2, wherein
the camera includes a light guide mechanism including an optical fiber.

5. The endoscope according to claim 4, wherein
in a horizontal cross-sectional view of the shaft, a proportion of a total area of all channels or lumens including the camera channel and the forceps channel to an area of the shaft is 65% or less.

6. The endoscope according to claim 5, wherein
in the horizontal cross-sectional view of the shaft, a proportion of a total area of all the channels or lumens in a semicircular portion (a remaining portion located in the semicircular portion when the channels and/or lumens partially protrude from the semicircular portion) on a side on which the camera channel is located to an area of the semicircular portion is 58% or less.

7. The endoscope according to claim 4, wherein
the shaft includes two water channels having a diameter (d3) of from 0.4 mm to 1.0 mm and four wire lumens having a diameter (d4) of from 0.2 mm to 0.5 mm formed therein,
an operation wire including a pullable tail end extends in each of the wire lumens, and
pulling the tail end deflects a distal end of the shaft.

8. The endoscope according to claim 7, further comprising:
a handle disposed on the proximal end side of the shaft and including a rotational operation portion;
a distal end tip made of resin, disposed on a distal end side of the shaft, and having an outer diameter substantially equal to that of the shaft, the distal end tip including a camera channel, a forceps channel, and water channels formed therein that are in communication with the camera channel, the forceps channel, and the water channels of the shaft, respectively, and that are open at a distal end surface of the distal end tip;
an intermediate member made of metal or made of ceramic, disposed between the shaft and the distal end tip, and having a plate shape with an outer diameter substantially equal to that of the shaft, the intermediate member including a main through-hole and four sub-through-holes, the main through-hole being formed surrounding and securing all of communication paths through which the camera channel, the forceps channel, and the water channels of the shaft communicates with the camera channel, the forceps channel, and the water channels of the distal end tip, respectively, the four sub-through-holes being formed corresponding to positions in which the wire lumens are formed; and
the four operation wires each passing through a respective one of the sub-through-holes and extending in a respective one of the wire lumens of the shaft, and each including a distal end large-diameter portion that is embedded in the distal end tip and that has a diameter larger than a diameter of the respective one of the sub-through-holes of the intermediate member.

9. The endoscope according to claim 1 that is used as a baby endoscope inserted into a forceps lumen of a side-view scope.

10. The endoscope according to claim 1 that is used for diagnostic treatment of a disease in a biliary duct or a pancreatic duct.
